(19) United States
(12) Patent Application Publication
(10) Pub. No.: US 2014/0065696 A1
(43) Pub. Date: Mar. 6, 2014

(54) Immunomodulatory Properties of Lactobacillus Strains

(71) Applicants: Delphine Saulnier, Bresse sur Grosne (FR); James Versalovic, Houston, TX (US); Bo Mollstam, Lerum (SE)

(72) Inventors: Delphine Saulnier, Bresse sur Grosne (FR); James Versalovic, Houston, TX (US); Bo Mollstam, Lerum (SE)

(21) Appl. No.: 14/074,480

(22) Filed: Nov. 7, 2013

Related U.S. Application Data
(62) Division of application No. 12/931,482, filed on Feb. 1, 2011.
(60) Provisional application No. 61/337,277, filed on Feb. 2, 2010.

(51) Int. Cl. A61K 35/74

(52) U.S. Cl. CPC A61K 35/74
USPC 435/252.9

(57) ABSTRACT

A specific method is provided of improving immunomodulatory properties of Lactobacillus strains using growth media with a specific primary carbon source, including a method of increasing the anti-inflammatory effect of non-pathogenic anti-inflammatory bacterial strains, by the use of specific growth conditions.

### CROSS-REFERENCE TO RELATED APPLICATIONS

This application claims priority from U.S. provisional application Ser. No. 61/337,277 filed Feb. 2, 2010.

### FIELD OF THE INVENTION

This invention relates to a method of increasing the immunomodulatory effect, for example anti-inflammatory effect, of certain bacterial strains of Lactobacillus spp., by the use of specific growth conditions, product formulations, products and methods using such bacteria for immunomodulatory purposes in a host, such as treatment and prophylaxis of inflammation caused by inflammation-causing agents.

### BACKGROUND OF THE INVENTION

The Food and Agricultural Organization of the United Nations define probiotics as “live microorganisms which when administered in adequate amounts confer a health benefit on the host”. Nowadays, a number of different bacteria are used as probiotics for example, lactic-acid producing bacteria such as strains of Lactobacillus and Bifidobacteria.

Prebiotics are defined as “non-digestible food ingredients that beneficially affect the host by selectively stimulating the growth and/or activity of one, or a limited number of bacteria in the colon that can improve the host health” Targets for prebiotics are usually bifidobacteria and lactobacilli. However, the selectivity of prebiotics is not always fully established, and hence stimulation of beneficial genera alone may be difficult to achieve. To alleviate the limitations of the probiotic and prebiotic approach, a solution could be to combine both in the form of a synbiotic.

Synbiotics were defined around a decade ago by Gibson and Roberfroid (1995) as “mixtures of pro- and prebiotics, which beneficially affect the host, by improving the survival and implantation of live microbial dietary supplements in the gastrointestinal tract (GT)”. The prebiotic should be a specific substrate for the probiotic, being able to stimulate its growth and/or activity while at the same time enhancing indigenous beneficial bacteria.

Lactic-acid producing bacteria are not only used for their beneficial effect on human or animal health, but they are also widely used in the food industry for fermentation processes. The effectiveness of probiotics is strain-specific, and each strain may contribute to host health through different mechanisms. Probiotics can prevent or inhibit the proliferation of pathogens, suppress production of virulence factors by pathogens, or modulate the immune response in a pro-inflammatory or an anti-inflammatory way. Use of different strains of the probiotic lactic-acid producing bacteria Lactobacillus reuteri is a promising therapy for the amelioration of infantile colic, alleviation of eczema, reduction of episodes of workplace illness, and suppression of Helicobacter pylori infection. L. reuteri is considered an indigenous organism of the human gastrointestinal tract and is present on the mucosa of the gastric corpus, gastric antrum, duodenum, and ileum. See, for example U.S. Pat. Nos. 5,439,678, 5,458,875, 5,534,253, 5,837,238, and 5,849,289. When L. reuteri cells are grown under anaerobic conditions in the presence of glycerol, they produce the antimicrobial substance known as reuterin (β-hydroxy propionaldehyde).

Monocytes leave the bone marrow and travel through the peripheral blood vessels until they reach the mucosa/serosa of the gastrointestinal tract. These putative macrophages are key to the interaction and propagation of the signals necessary to regulate the immune system. In the gastrointestinal tract for example, there is a constant level of immune response in the macrophages of the mucosal epithelium to the bacteria in the intestinal lumen and attached to the intestinal mucosa. In the normal state, this response involves the generation of cytokine signals to restrict and contain an unnecessary inflammatory response. However, when a pathogen or toxin is presented to these cells, they form the first line of defense and react by producing an increasing amount of pro-inflammatory cytokines, which propagate the inflammatory response until the threat is removed. The generation of cytokines relevant to the interactions with commensal (non-threatening) bacteria as well as those involved in the full inflammatory response to pathogens is subject to intervention by lactic acid bacteria themselves (including surface antigens) or by substances produced by these lactic acid bacteria and it is clear that the commensal flora has extensive interaction with the macrophages of the mucosa to maintain a balanced reaction to the gut flora and thereby maintain optimal health.

It is known that various pathogens can cause inflammation, for example in the gastrointestinal tract. Such inflammation, for example, in the stomach and gastrointestinal tract, is mediated by intercellular signal proteins known as cytokines, which are produced by macrophages and dendritic cells in the epithelium in response to an antigenic stimulus such as that produced by a pathogen. Upon contact between the epithelium and the antigen of a pathogen or endotoxins produced by it, such as lipopolysaccharides (LPS), antigen presenting cells (including dendritic cells) in the epithelium propagate the signal to naive macrophages which then respond in a so-called Th-1 type response where pro-inflammatory cytokines including TNFα, IL-1, IL-6, IL-12 are produced by the macrophages. These cytokines in turn stimulate natural killer cells, T-cells and other cells to produce interferon γ (IFNγ), which is the key mediator of inflammation. IFNγ leads to an escalation of the inflammatory response and the reactions described above that lead to cytotoxicity. Naive macrophages can also respond to antigens with a Th-2 type response. This response is suppressed by IFNγ. These Th-2 type cells produce anti-inflammatory cytokines such as IL-4, IL-5, IL-9 and IL-10. IL-10 is known to inhibit the production of IFNγ and thus dampen the immune response. The balance between Th-1 and Th-2 type cells and their respective cytokine production defines the extent of the inflammation response to a given antigen. Th-2 type cells can also stimulate the production of immunoglobulins via the immune system. Anti-inflammatory activity in the gastrointestinal tract, where there is a reduced TNFα level, correlates with enhanced epithelial cells (gut wall lining integrity) and thus to a reduction in the negative effects caused by gastrointestinal pathogens and toxins.

Inflammation can be involved in several diseases in mammals both externally, for example on skin and eyes, and internally for example on various mucous membranes, in the mouth, gastrointestinal (GI) tract, vagina etc. but also in muscles, bone-joints, cardio vascular organs and tissues, including blood vessels and in brain-tissue and the like. In the GI tract there are several diseases connected to inflammation, for example, gastritis, ulcers, and inflammatory bowel disease (IBD). The disease has been linked to imbalances in the gut microflora and an over-expressed inflammatory reaction to components of the normal gut flora and this reaction is currently treated with poor success using a series of different drugs, one of which is based on anti-TNFα therapy designed to reduce the levels of TNFα in the gastro-intestinal mucosa. There are also several other diseases associated with inflammation, such as gingivitis, vaginitis, atherosclerosis, and various cancer forms that are thought to be associated with the composition of the microflora in different localities of the body.

With the shortcomings of abovementioned anti-TNFα therapies in mind it was a positive surprise when the inventors of the invention herein demonstrated that the replacement of sucrose by glucose as the sole carbon source for the growth of specific anti-inflammatory strains of L. reuteri in a defined medium significantly inhibited the TNFα production in LPS-stimulated macrophages. Accordingly growing specific anti-inflammatory Lactobacillus strains with a defined carbon source such as glucose gives the opportunity to provide anti-inflammatory strains of L. reuteri with even more potent anti-inflammatory properties.

As can be understood from the invention herein, also pro-inflammatory bacterial strains can be modified in their immunomodular properties, by modifying the carbon source for the bacteria to grow in.

As mentioned before, anti-inflammatory activity has already been associated with various lactobacilli for example U.S. Pat. No. 7,105,336 B2 describes Lactobacillus strains selected for their ability to reduce gastrointestinal inflammation associated with H. pylori infection in mammals using a mouse macrophage assay for TNFα activity. Another patent application mentioning the anti-inflammatory activity of L. reuteri is US 2008/0254011 A1 describing strains of lactic acid bacteria selected for their capability of increasing the BSH-activity and consequently lowering serum LDL-cholesterol, and simultaneously decreasing the pro-inflammatory cytokine TNF-alpha levels for the treatment of cardiovascular diseases.

US 2006/0233775 A1 describes the selection of strains of lactic acid bacteria selected for their capability of reducing inflammation, such as intestinal bowel disease. However none of above mentioned inventions attaches an importance to the choice of carbon source for the decrease in TNFα production.

Regulating other different activities of Lactobacillus spp. by the choice of different sugars is already known in the art. For example, Ávila et al (2009) showed that the growth condition was important for the regulation of α-L-rhamnosidase, as the activity was down regulated by the addition of glucose.

Growth behavior on glucose and sucrose has been studied by Årsköld et al (2008). It was shown that the choice of sugar source clearly affected the growth performance of L. reuteri ATCC 55730. Growth on sucrose resulted in a high growth rate and an appropriate biomass yield, whereas growth with glucose was characterized by a maximum specific growth rate and low ATP levels.

However nobody has hitherto demonstrated that specific carbon sources in the growth media can regulate the TNFα production. It is therefore one object of the invention to enhance the anti-inflammatory effect of already anti-inflammatory strains of L. reuteri as seen for example by reduced TNFα production in a host. It is a further object of the invention to provide products containing said strains, including agents for treatment or prophylaxis of inflammation caused by inflammation-causing agents for administration to humans, including conditioned media in which said strains have grown and protein-containing extracts thereof.

Another object of the invention is to provide products containing said strains together with a specific carbon source, in order to have a synbiotic product. A further object of this invention is to provide specific carbon source, such as sugar for consumption to individuals already colonized with anti-inflammatory strains of lactic acid bacteria.

Other objects and advantages will be more fully apparent from the following disclosure and appended claims.

### SUMMARY OF THE INVENTION

The invention herein provides a specific method of improving immunomodulatory properties of lactic acid bacterial strains using growth media with a specific carbon source, including a method of increasing the anti-inflammatory effect of nonpathogenic anti-inflammatory bacterial strains, by the use of specific growth conditions.

A primary object of the present invention is to increase the immunomodulatory effect in mammals, of certain bacterial strains of lactic acid bacteria, by the use of specific growth conditions.

It is another object to enhance the anti-inflammatory effect of anti-inflammatory strains of L. reuteri by the choice of carbon source in the growth media.

Another object of the invention is to enhance the anti-inflammatory effect, in mammals seen as a decreased TNF-α production, of an anti-inflammatory L. reuteri strain together with glucose, lactose, fructose, starch, 1,2 propanediol or a prebiotic such as fructooligosaccharides as a primary carbon source in the growth media.

It is a further object of the invention to provide products containing said strains.

It is a further object of the invention to provide products containing said strains together with a specific carbon source, in order to have a synbiotic product.

Another object of this invention is to provide specific carbon sources in such a way that they are not digestible in the gastrointestinal tract such as prebiotic for consumption to individuals already colonized with anti-inflammatory strains of L. reuteri.

Other objects and advantages of the present invention will become obvious to the reader and it is intended that these objects and advantages are within the scope of the present invention.

### BRIEF DESCRIPTION OF THE DRAWINGS

FIG. 1 is a graph showing how conditioned media with L. reuteri ATCC 5289 grown with different sugar as sole carbon sources effect TNF-α inhibition.

FIG. 2 is a graph showing how L. reuteri conditioned media increases TNF-α production if glucose is replaced by sucrose (in LDMIII medium).

FIG. 3 is a graph showing how conditioned media with L. reuteri DSM 17938 and ATCC PTA 6475 grown with different carbon sources effect TNF-α inhibition.

FIG. 4 is a table showing whether different carbon sources together with L. reuteri are capable of inhibiting TNFα production, (+) indicates inhibition of TNFα production.

### DETAILED DESCRIPTION OF THE INVENTION AND PREFERRED EMBODIMENTS THEREOF

Lactobacillus reuteri is a heterofermentative lactic acid bacterial species that naturally inhabits the gut of humans and animals. Specific probiotic L. reuteri strains potently suppress human TNFα production while other probiotic L. reuteri strains enhance human TNFα production.

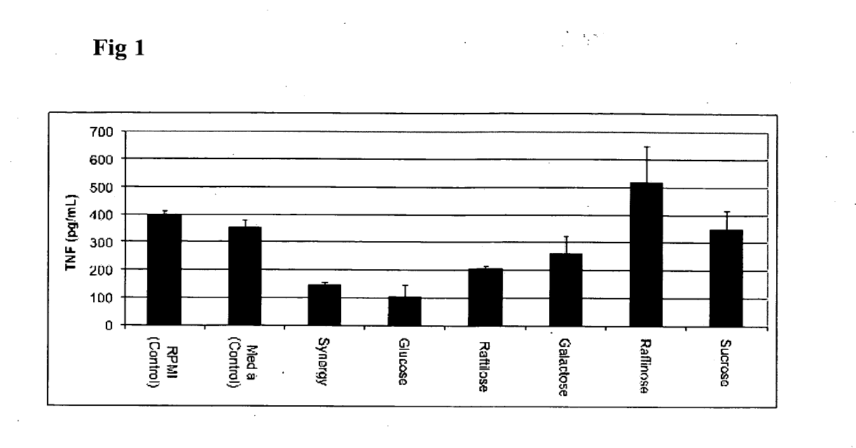

In order to show how anti-inflammatory strains of L. reuteri grown with different carbon sources effect TNFα production, L. reuteri (ATCC 5289) was grown anaerobically in a defined medium with different sugars as sole carbon source until late stationary phase. Surprisingly, growth on glucose as the carbon source significantly decreased the production of TNF-α compared to growth on sucrose. The results are shown in FIG. 1.

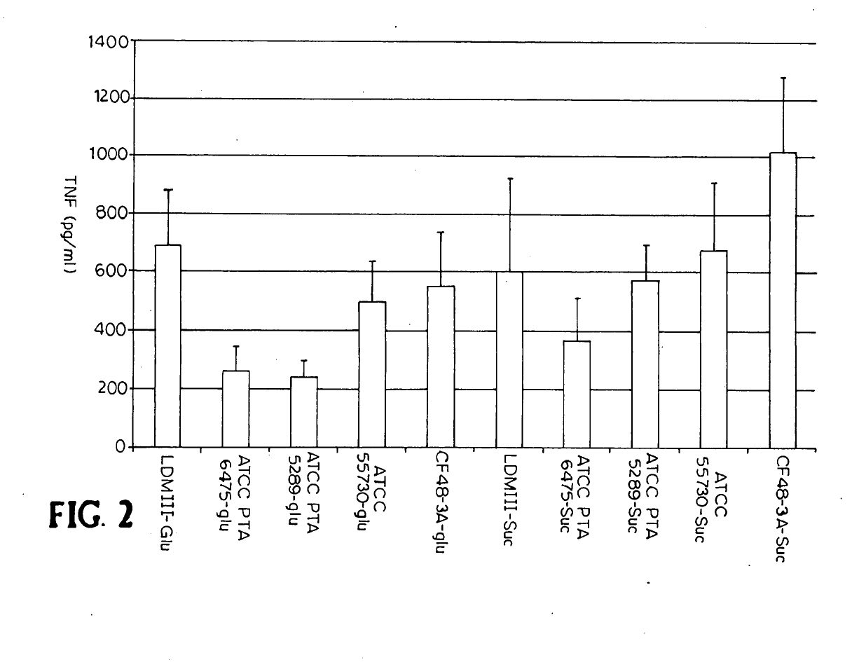

A study was performed to identify how glucose and sucrose in the growth media of different strains of L. reuteri influence on TNF-α production. Strains of L. reuteri already known to be TNF-inhibitory (ATCC PTA 6475 and ATCC PTA 5289) or TNF-stimulatory (ATCC 55730 and CF483A) were grown anaerobically in a defined medium with glucose or sucrose as sole carbon source until late stationary phase (24-28 hours). These results show that growth using sucrose as the primary carbon in for example the strains L. reuteri ATCC PTA-6475 and L. reuteri ATCC PTA-5289 significantly increased the production of LPS-stimulated TNF-α in human cells (FIG. 2) compared with growth on glucose.

The results showed in FIG. 2 also raise the idea of the possibility to affect for example pro-inflammatory strains of L. reuteri by the growth media, i.e. making them more inflammatory. Increased inflammatory properties could be useful in certain disease conditions, for example cancer or allergy prevention.

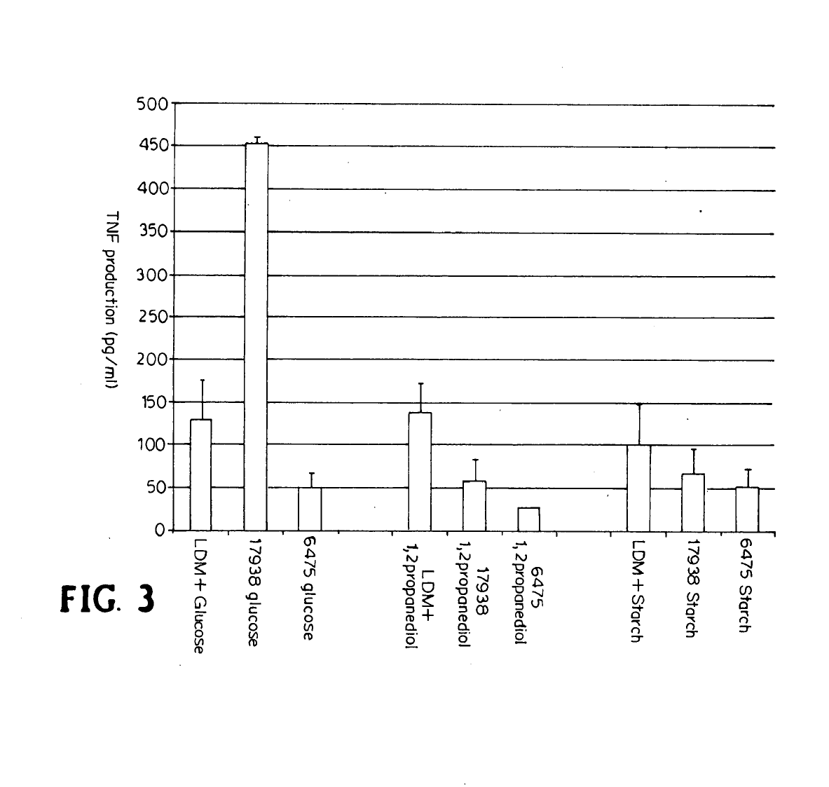

The effect of other carbon sources was also studied. L. reuteri (ATCC PTA 6475, DSM 17938) was grown anaerobically in a defined medium with glucose, 1,2 propanediol or starch as sole carbon source until late stationary phase. These results showed that DSM 17938 becomes TNF inhibitory when grown on 1,2 propanediol or starch as sole carbon source and ATCC PTA 6475 showed similar results for all three carbon sources (FIG. 3).

Further this increased TNF-inhibitory effect is also seen when L. reuteri is grown in defined medium with lactose or fructose as sole carbon source (data not shown).

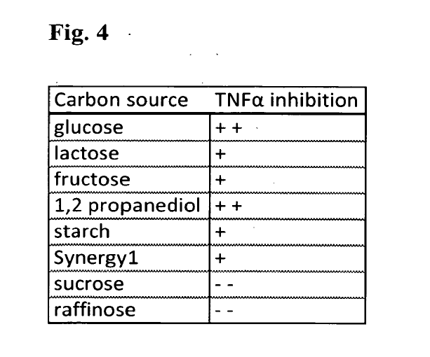

The strains of L. reuteri grown in defined medium with glucose, lactose, fructose, starch or 1,2 propanediol that are capable of decreasing the TNFα production include but are not limited to ATCC PTA 6475, ATCC PTA 5289, ATCC 4659, JCM 1112, and DSM 20016. A list of carbons sources that will affect L. reuteri to decrease TNFα production can be seen in FIG. 4.

Products containing strains or conditioned medium capable of decreasing TNF-α production can be supplemented with specific carbon sources for example glucose after freeze-drying, in order to have a synbiotic product.

Carbon sources that together with lactic acid bacteria are capable of decreasing the TNFα production are preferably but not limited to glucose, lactose, fructose, starch, 1,2 propanediol or a prebiotic such as fructooligosaccharides with different degree of polymerization for example Synergy 1® (mixture of fructooligosacharides and inulin, Orafti).

The product is preferably formulated but not limited to a tablet or a capsule.

The carbon source is integrated in the product in such a way that it will not be digested in the gastrointestinal tract, for example the glucose can be encapsulated separately in microcapsules as known in the art, before integrated in the tablet or capsule, with the chosen Lactobacillus strain.

The specific carbon source such as encapsulated glucose can be consumed by individuals known to be already colonized with anti-inflammatory strains, for example L. reuteri.

Since numerous modifications and changes will readily occur to those skilled in the art, it is not desired to limit the invention to the exact construction and operation shown and described, and accordingly, all suitable modifications and equivalents may be resorted to, falling within the scope of the invention.

### EXAMPLE 1

Conditioned media with L. reuteri ATCC 5289 grown with different sugar sources effect TNFα inhibition

THP-1 cells were incubated together with conditioned media (CM) from the growth of L. reuteri ATCC 5289. The conditioned media are cell-free supernatants from 24-hour cultures of L. reuteri ATCC 5289 cultured in LDMIII (S. Jones and J. Versalovic, BMC Microbiol. 2009; 9: 35) supplemented with one specific sugar as sole carbon source. THP-1 cells were stimulated with either control medium or E. coli-derived LPS (which leads to the generation of TNF-α in a normal inflammatory response) or PCK during a 3.5 hour incubation after which the cells were removed and the supernatants assayed for TNFα levels using an ELISA technique as known in the art.

Materials and Methods:

Key reagents, bacterial strains and mammalian cell lines

L. reuteri strains were grown in deMan, Rogosa, Sharpe (MRS; Difco, Franklin Lakes, N.J.) or LDMIII (pH 6.5) with a unique sugar source (see list of sugar source used below) for LDM medium composition). An anaerobic chamber (1025 Anaerobic System, Forma Scientific, Waltham, Mass.) supplied with a mixture of 10% CO₂, 10% H₂, and 80% N₂ was used for anaerobic culturing of lactobacilli.

Biogaia AB (Raleigh, N.C.) provided L. reuteri strains ATCC PTA 5289. THP-1 cells (ATCC TIB-202) were maintained in RPMI 1640 supplemented with 10% fetal bovine serum (Invitrogen, Carlsbad, Calif.) at 37° C. and 5% CO₂. All chemical reagents were obtained from Sigma-Aldrich (St Louis, Mo.) unless otherwise stated. Polystyrene 96- and 24-well plates for biofilm and tissue culture studies were obtained from Corning (Corning, N.Y.). Filters with polyvinylidene fluoride membranes (0.22 mm pore size) (Millipore, Bedford, Mass.) were used for sterilization.

Sugar sources used in LDMIII medium:
D-Glucose (G8270, >99.5% glucose, Sigma)
Sucrose (S9378, 99.9% sucrose, Sigma)
D-Galactose (G0750, >99% galactose, Sigma)
Raffinose (R0260, >98% raffinose, Sigma)
Fructooligosaccharides, Raftilose, (Orafti®P95, average degree of polymerization=11, 92% fructooligosaccharides, 8% glucose, fructose and sucrose, Beneo Orafti)
1:1 Mixture of fructooligosacharides and inulin (Orafti®Synergyl, 92% fructooligosaccharides and inulin, 8% glucose, fructose and sucrose, Beneo Orafti) Preparation of cell-free supernatants from L. reuteri cultures for immunomodulation studies

For immunomodulation studies, 10 mL of LDMIII with a unique carbon source was inoculated with L. reuteri cultures (grown overnight in MRS broth for 16-18 hrs) and adjusted to OD600=0.1. Bacteria were then incubated for 24 hours at 37° C. in anaerobic conditions. Final OD600 was measured and LDM was diluted to the same OD for all samples to take into account possible growth variation with different sugar source. Cells were pelleted (4000×g, RT, 10 minutes) and discarded. Supernatants were filter-sterilized (0.22 μm pore size). Aliquots were vacuum-dried and resuspended to the original volume using RPMI.

TNF inhibition experiments

As previously described (Lin et al, 2008), cell-free supernatants of L. reuteri planktonic cell (5% v/v) and E. coli 0127:B8 LPS (100 ng/mL) were added to human THP-1 cells (approximately 5×104 cells). Plates were incubated at 37° C. and 5% CO2 for 3.5 hours. THP-1 cells were pelleted (1500×g, 5 minutes, 4° C.), and TNF quantities in monocytoid cell supernatants were determined by quantitative ELISAs (R&D Systems, Minneapolis, Minn.). RPMI and Media are both used as controls (typically RPMI 95% and Media=LDM 5%).

Results

Addition of LPS to the THP-1 cells in the presence of 1) Synergyl (BENEO-Orafti Inc. 2740 Route 10 West Morris Plains, N.J. 07950, USA); 2) Glucose; 3) Raftilose; 4) Galactose; 5) Raffinose; and 6) Sucrose led to the generation of 1) 145.3 pg/ml TNFα, 2) 104.3 pg/ml TNFα, 3) 204.3 pg/ml TNFα, 4) 260 pg/ml TNFα 5) 517.8 pg/ml TNFα, and 6) 347.9 pg/ml TNFα, respectively, during the 3.5 hour incubation period. Addition of the growth medium (RPMI and LDM) which acts as a control for the CM additions, led to the generation of 396 and 352 pg/ml TNFα respectively. The results show that glucose and or Synergyl as a sole carbon source inhibited the TNFα production more than 50% compared to the RPMI and LDM controls. This was not the case when the strain was grown with sucrose or raffinose. The results are shown in FIG. 1. LDM+ each sugar alone without the strains was also tried to make sure that the sugars were not responsible directly with the change in TNF response (results not shown).

### EXAMPLE 2

Conditioned media with strains of L. reuteri already known to be TNF-inhibitory (ATCC PTA 6475 and ATCC PTA 5289) or TNF-stimulatory (ATCC 55730 and CF483A) grown with different sugar sources effect TNF-α inhibition

THP-1 cells were incubated together with conditioned media (CM) from the growth of selected L. reuteri strains grown with glucose, L. reuteri ATCC PTA-6475, L. reuteri ATCC PTA-5289, L. reuteri ATCC 55730 and L. reuteri strain CF48-3A and the same strains grown with sucrose. THP-1 cells were stimulated with either control medium (LDMIII) or E. coli-derived LPS during a 3.5 hour incubation after which the cells were removed and the supernatants assayed for TNFα levels using an ELISA technique. LDMIII with glucose respectively sucrose was used as a control.

The materials and methods were as in Example 1.

Results

The results show (see FIG. 2) that L. reuteri conditioned medium of the two anti-inflammatory strains L. reuteri ATCC PTA-6475 and L. reuteri ATCC PTA-5289 increases TNF-α production if glucose is replaced by sucrose in LDMIII.

### EXAMPLE 3

Formulation of the conditioned medium of L. reuteri ATCC PTA-5289 grown in glucose as a sole carbon source.

Using the method in Example 1, the conditioned medium from one effectively TNF-α decreasing strain was selected, in this example the medium from L. reuteri ATCC PTA-5289 grown with glucose as a sole carbon source. This medium was produced in larger scale by growing the strain in de Man, Rogosa, Sharpe (MRS) (Difco, Sparks, Md.). Overnight cultures of lactobacilli were diluted to an OD₆₀₀ of 1.0 (representing approximately 10⁹ cells/ml) and further diluted 1:10 and grown for an additional 24 h. Bacterial cell-free conditioned medium was collected by centrifugation at 8500 rpm for 10 min at 4° C. Conditioned medium was separated from the cell pellet and then filtered through a 0.22 μm pore filter unit (Millipore, Bedford, Mass.). The conditioned medium was then lyophilized and formulated, using standard methods, to make a tablet.

### EXAMPLE 4

Formulation of freeze dried L. reuteri ATCC PTA-5289 powder in a standard manner, supplemented with glucose after freeze-drying.

Fermentation medium composition:

Dextrose mono hydrate 60 g/l

Yeast extract KAV 20 g/l

Peptone Type PS (of pig origin) 20 g/l

Di ammonium hydrogen citrate 5 g/l

Sodium acetate (×3 H₂O) 4.7 g/l

Di potassium hydrogen phosphate 2 g/l

Tween80 0.5 g/l

Silibione (anti foam) 0.14 g/l

Magnesium sulphate 0.10 g/l

Manganese sulphate 0.03 g/l

Zinc sulphate hepta hydrate 0.01 g/l

Water q.s.

Centrifuge medium

Peptone O-24 Orthana (of pig origin)

Cryoprotectants;

Lactose (of bovine origin) 33%

Gelatin hydrolysate (of bovine origin) 22%

Sodium glutamate 22%

Maltodextrin 11%

Ascorbic acid 11%

Production steps of freeze dried lactic acid bacteria powder
1. Twenty ml of the media is inoculated with 0.6 ml of freeze-dried lactic acid bacteria powder from a working cell bank vial. The fermentation is performed in a bottle at 37° C. for 18-20 hours without stirring or pH control i.e. static.
2. Two 1-liter flasks of the media are inoculated with 9 ml cell slurry per liter. The fermentation is performed at 37° C. for 20-22 hours without stirring or pH control i.e. static.
3. The two one liter cell slurries from step no. 2 inoculates the 600-liter vessel. The fermentation is performed at 37° C. for 13 hours with stirring and pH control. At the start of the fermentation the pH is 6.5. The pH control starts when the pH drops below 5.4 using a 20% sodium hydroxide solution. The pH control is set to pH 5.5.
4. The fourth and final fermentation step is performed in a 15000-liter vessel with the inoculation from step no 3. The fermentation is performed at 37° C. for 9 to 12 hours with stirring and pH control. At the start of the fermentation the pH is 6.5. The pH control starts when the pH drops below 5.4 using a 20% sodium hydroxide solution. The pH control is set to pH 5.5. The fermentation is complete when the culture reaches the stationary phase, which can be seen by the reduction of the addition of the sodium hydroxide solution. Roughly 930 liters of the sodium hydroxide solution is added to the 10200 liters of media and 600 liters of inoculum during the fermentation.

5. The cell slurry from the final fermentation is separated at 10° C. twice in a continuous centrifuge from Alfa Laval. After the first centrifugation the volume of the cell slurry is reduced from roughly 11730 liters to 1200 liters. This volume is washed with 1200 liters of a peptone (Peptone O-24, Orthana) solution in a 3000-liter vessel and is separated again before the mixing with the cryoprotectants. The washing step with peptone is performed to avoid any freezing-point reduction in the freeze-drying process.
6. After the second centrifugation the volume of the cell slurry is reduced to 495 liters. This volume is mixed with 156 kg of the cryoprotectant solution to reach roughly 650 liters of the cell slurry.
7. The cell slurry is pumped to a 1000-liter vessel. The vessel is then transported to the freeze-drying plant.
8. At the freeze-drying plant, exactly 2 liters of the cell slurry is poured on each plate in the freeze dryer. The maximum capacity of the freeze dryer is 600 liters and all excessive cell slurry volume is thrown away.
9. The cell slurry of Lactobacillus reuteri has a dry matter content of 18% and is freeze-dried for four to five days.
10. During the freeze-drying process, the pressure in the process is between 0.176 mbar and 0.42 mbar. The vacuum pump is started when the pressure reaches 0.42 mbar. The PRT (pressurizing test) is used to determine when the process is complete. If the PRT or the increase of pressure is less then 0.02 mbar after 120 seconds, the process is stopped.

The freeze-dried Lactobacillus reuteri was then supplemented with glucose and formulated, using standard methods, to make a tablet or capsule for example as described in Example 6.

### EXAMPLE 5

Formulation of freeze-dried L. reuteri ATCC PTA-5289 with sucrose as the sole carbon source.

This was done as described in Example 4 but with sucrose as a sole carbon source instead of dextrose during fermentation.

The freeze-dried Lactobacillus reuteri was then formulated, using standard methods, to make a tablet or capsule for example as described in Example 6 (but without the addition of glucose, paragraph 4).

### EXAMPLE 6

Manufacturing of products containing selected strain

In this example, L. reuteri (ATCC PTA-5289) is selected based on good anti-inflammatory characteristics in general and TNF-inhibiting properties in order to add the strain to a tablet. The L. reuteri strain is grown and lyophilized, using standard methods for growing Lactobacillus in the industry as can be read in Example 4.

The steps of an example of a manufacturing process of tablet containing the selected strain follow including encapsulated glucose, with it being understood that excipients, fillers, flavors, encapsulators, lubricants, anticaking agents, sweeteners and other components of tablet products as are known in the art, may be used without affecting the efficacy of the product:
1 Melting. Melt SOFTISAN™ 154 (SASOL GMBH, Bad Homburg, Germany) in a vessel and heat it to 70 ° C. to assure complete disruption of the crystalline structure. Then cool it down to 52-55° C. (just above its hardening point).
2 Granulation. Transfer Lactobacillus reuteri freeze-dried powder to a Diosna high-shear mixer/granulator, or equivalent. Add slowly during approximately 1 minute the melted SOFTISAN™ 154 to the Lactobacillus reuteri powder. Use chopper during the addition.
3 Wet-sieving. Immediately after the granulation, pass the granules through a 1-mm sieving net by using a Tornado mill. The sieved granulate is packed in alupouches, made out of PVC-coated aluminum foil, sealed with a heatsealer to form a pouch, together with desiccant pouch, and stored refrigerated until mixing. The granulated batch is divided for two tablet batches.
4. Add encapsulated D-Glucose (G8270, >99.5% glucose, Sigma), encapsulated using standard microencapsulating methods as known in the art. The amount of sugar is dependent on the total CFU of the added powder of dry L. reuteri, a standard level can be 1 gram of sugar per total CFU of 1E+08 of bacteria but this could also be varied down to 0.1 gram or 0.01 gram up to 10 gram even up to 100 gram of sugar.
5 Mixing. Mix all the ingredients in a mixer, to a homogenous blend.
6 Compression. Transfer the final blend to the hopper of a rotary tablet press and compress tablets with a total weight of 765 mg, in a Kilian compressor.

7 Bulk packaging. The tablets are packed in alu-bags together with a drying pouch of molecular sieve. The alu-pouch is put in a plastic bucket and stored in a cool place at least one week, before final package.

The use of SOFTISAN™, a hydrogenated palm oil, enables the Lactobacillus cells to be encapsulated in fat and environmentally protected.

As stated above, the product of the invention may be in forms other than tablet, and standard methods of preparing the underling underlying product as are known in the art are beneficially used to prepare the product of the invention including the selected L. reuteri culture.

### EXAMPLE 7

A female subject suffering from colitis is treated with the product produced in example 4. The subject is treated twice daily, in the morning and at night.

After two weeks, the inflammation of the colon is significantly decreased. On cessation of L. reuteri treatment the condition returns but is suppressed with regular administration of L. reuteri.

While the invention has been described with reference to specific embodiments, it will be appreciated that numerous variations, modifications, and embodiments are possible, and accordingly, all such variations, modifications, and embodiments are to be regarded as being within the spirit and scope of the invention.

1. (canceled)

2. A method for enhancing the anti-inflammatory effects of anti-inflammatory lactic acid bacteria by growing the lactic acid bacteria on a specific carbon source selected from the group consisting of glucose, lactose, fructose, starch and 1,2-propanediol and administering the lactic acid bacteria to a mammal.

3. A method of enhancing anti-inflammatory effects of an anti-inflammatory strain of Lactobacillus reuteri comprising growing the strain of Lactobacillus reuteri in a medium comprising a carbon source selected from a group consisting of glucose, lactose, fructose, starch and 1,2-propanediol and administering the strain of Lactobacillus reuteri to a mammal.

4. The method of claim 3, where the Lactobacillus reuteri strain is ATCC PTA 6475.

5. The method of claim 3, where the Lactobacillus reuteri strain is ATCC PTA 5289.

6. (canceled)

7. (canceled)

8. (canceled)

9. (canceled)
